(19) United States
(12) Patent Application Publication
(10) Pub. No.: US 2024/0365737 A1
(43) Pub. Date: Nov. 7, 2024

(54) ARUGULA VARIETY 'SPARTAN'

(71) Applicant: Pinnacle Seed, Inc., Carmel, CA (US)

(72) Inventor: Christopher H. BECKER, Mount Vernon, WA (US)

(73) Assignee: Pinnacle Seed, Inc., Carmel, CA (US)

(21) Appl. No.: 18/648,224

(22) Filed: Apr. 26, 2024

Related U.S. Application Data
(60) Provisional application No. 63/499,434, filed on May 1, 2023.

(51) Int. Cl. A01H 6/20; A01H 5/12

(52) U.S. Cl. CPC A01H 6/20; A01H 5/12

(57) ABSTRACT

New arugula variety designated ‘Spartan’ is described. ‘Spartan’ exhibits stability and uniformity.

### CROSS-REFERENCE TO RELATED APPLICATION

This application claims the benefit of U.S. Provisional Application No. 63/499,434, filed May 1, 2023, which is hereby incorporated by reference in its entirety.

### FIELD

The present invention relates to the field of plant breeding. In particular, this invention relates to new arugula (Eruca sativa Mill.) variety ‘Rock1’ and to new arugula (Diplotaxis tenuifolia Mill.) variety ‘Spartan’.

### BACKGROUND

Eruca sativa and Diplotaxis tenuifolia, both known as arugula, are a specialty leaf vegetable, also known as wild rocket, sand mustard, sand rocket, Lincoln's weed, white rocket, and wall rocket.

Arugula is an important and valuable vegetable crop. As a result of this demand, there is a continued need for new arugula varieties. In particular, there is a need for improved flat arugula varieties that are stable, high yielding, and agronomically sound.

### SUMMARY

In order to meet these needs, the present invention is directed to improved arugula varieties.

In one aspect, the present invention is directed to Eruca sativa Mill. arugula seed designated as ‘Rock1’, representative sample of seed having been deposited under ATCC Accession Number X1. In another embodiment, the present invention is directed to an arugula plant and parts thereof produced by growing Eruca sativa Mill. arugula seed designated as ‘Rock1’, representative sample of seed having been deposited under ATCC Accession Number X1. Arugula plant parts include arugula leaves, ovules, pollen grains, seeds, embryos, embryo sacs, fruits, cells, calli, protoplasts, roots, rootstocks, stems, meristems, cotyledons, hypocotyls, flowers, ovaries, pericarps, anthers, microspores, and the like, and portions thereof. In another embodiment, the present invention is directed to an arugula plant and parts thereof having all the physiological and morphological characteristics of an arugula plant and parts thereof produced by growing ‘Rock1’ Eruca sativa Mill. arugula seed having ATCC Accession Number X1. In another embodiment, the present invention is directed to an F₁ hybrid arugula plant having ‘Rock1’ as a parent, where ‘Rock1’ is grown from ‘Rock1’ Eruca sativa Mill. arugula seed having ATCC Accession Number X1. In another embodiment, the present invention is directed to a tissue culture produced from protoplasts or cells of an arugula plant and a cell culture produced from protoplasts or cells of an arugula plant, and an arugula plant regenerated from said tissue or cell culture, wherein the arugula plant is produced by growing ‘Rock1’ Eruca sativa Mill. arugula seed having ATCC Accession Number X1. In still another embodiment, the present invention relates to a plant regenerated from a tissue culture produced from protoplasts or cells of an arugula plant, wherein the arugula plant is produced by growing ‘Rock1’ Eruca sativa Mill. arugula seed having ATCC Accession Number X1.

In another embodiment, the present invention relates to a method of making arugula seeds including crossing an arugula plant produced by growing ‘Rock1’ Eruca sativa Mill. arugula seed having ATCC Accession Number X1 with another arugula plant and harvesting seed therefrom. In some embodiments, the method further includes (a) selecting one or more of the F₁ progeny for plants that have a desired trait to produce selected progeny plants; (b) crossing the selected progeny plants with a variety of arugula plant designated ‘Rock1’, representative sample of seed of ‘Rock1’ having been deposited under ATCC Accession Number X1, to produce backcross progeny plants; (c) selecting for backcross progeny plants that have the desired trait and all of the physiological and morphological characteristics of ‘Rock1’ arugula when grown in the same environmental conditions to produce selected backcross progeny plants; and (d) repeating steps (b) and (c) three or more times in succession to produce selected fourth or higher backcross progeny plants that comprise the desired trait and all of the physiological and morphological characteristics of ‘Rock1’ arugula when grown in the same environmental conditions.

In another aspect, the present invention relates to a method of producing a seed of a ‘Rock1’-derived arugula plant, comprising crossing an Eruca sativa Mill. plant designated as ‘Rock1’, representative sample of seed having been deposited under ATCC Accession Number X2, with a second Eruca sativa Mill. plant, whereby seed of a ‘Rock1’-derived arugula plant forms. In some embodiments, the method further includes (a) crossing a plant grown from Eruca sativa Mill. seed derived from ‘Rock1’ with itself or with a second Eruca sativa Mill. plant to yield additional seed of a ‘Rock1’-derived arugula plant; (b) growing the additional seed of a ‘Rock1’-derived arugula plant of step (a) to yield additional ‘Rock1’-derived arugula plants; and (c) repeating the crossing and growing of steps (a) and (b) for an additional 3-10 generations to generate further ‘Rock1’-derived arugula plants.

In another embodiment, the present invention relates to a method of producing an arugula leaf from the plant of any of the above embodiments, said method comprising growing the plant of the above embodiments until it produces at least one leaf, and harvesting the leaf.

In another aspect, the present invention is directed to Diplotaxis tenuifolia Mill. arugula seed designated as ‘Spartan’, representative sample of seed having been deposited under ATCC Accession Number X2. In one embodiment, the present invention is directed to an arugula plant and parts thereof produced by growing Diplotaxis tenuifolia Mill. arugula seed designated as ‘Spartan’, representative sample of seed having been deposited under ATCC Accession Number X2. Arugula plant parts include arugula leaves, ovules, pollen grains, seeds, embryos, embryo sacs, fruits, cells, calli, protoplasts, roots, rootstocks, stems, meristems, cotyledons, hypocotyls, flowers, ovaries, pericarps, anthers, microspores, and the like, and portions thereof. In another embodiment, the present invention is directed to an arugula plant and parts thereof having all the physiological and morphological characteristics of an arugula plant and parts thereof produced by growing ‘Spartan’ Diplotaxis tenuifolia Mill. arugula seed having ATCC Accession Number X2. In another embodiment, the present invention is directed to an F₁ hybrid arugula plant having ‘Spartan’ as a parent, where ‘Spartan’ is grown from ‘Spartan’ Diplotaxis tenuifolia Mill. arugula seed having ATCC Accession Number X2. In another embodiment, the present invention is directed to a tissue culture produced from protoplasts or cells of an arugula plant and a cell culture produced from protoplasts or cells of an arugula plant, and an arugula plant regenerated from said tissue or cell culture, wherein the arugula plant is produced by growing ‘Spartan’ Diplotaxis tenuifolia Mill. arugula seed having ATCC Accession Number X2. In still another embodiment, the present invention relates to a plant regenerated from a tissue culture produced from protoplasts or cells of an arugula plant, wherein the arugula plant is produced by growing ‘Spartan’ Diplotaxis tenuifolia Mill. arugula seed having ATCC Accession Number X2.

In another embodiment, the present invention relates to a method of making arugula seeds comprising crossing an arugula plant produced by growing ‘Spartan’ Diplotaxis tenuifolia Mill. arugula seed having ATCC Accession Number X2 with another arugula plant and harvesting seed therefrom, and optionally further comprising (a) selecting one or more of the F₁ progeny for plants that have a desired trait to produce selected progeny plants; (b) crossing the selected progeny plants with a variety of arugula plant designated ‘Spartan’, representative sample of seed of ‘Spartan’ having been deposited under ATCC Accession Number X2, to produce backcross progeny plants; (c) selecting for backcross progeny plants that have the desired trait and all of the physiological and morphological characteristics of ‘Spartan’ arugula when grown in the same environmental conditions to produce selected backcross progeny plants; and (d) repeating steps (b) and (c) three or more times in succession to produce selected fourth or higher backcross progeny plants that comprise the desired trait and all of the physiological and morphological characteristics of ‘Spartan’ arugula when grown in the same environmental conditions.

In another aspect, the present invention relates to a method of producing a seed of a ‘Spartan’-derived arugula plant, comprising crossing an Diplotaxis tenuifolia Mill. plant designated as ‘Spartan’, representative sample of seed having been deposited under ATCC Accession Number X2, with a second Diplotaxis tenuifolia Mill. plant, whereby seed of a ‘Spartan’-derived arugula plant forms; and optionally further comprising (a) crossing a plant grown from Diplotaxis tenuifolia Mill. seed derived from ‘Spartan’ with itself or with a second Diplotaxis tenuifolia Mill. plant to yield additional seed of a ‘Spartan’-derived arugula plant; (b) growing the additional seed of a ‘Spartan’-derived arugula plant of step (a) to yield additional ‘Spartan’-derived arugula plants; and (c) repeating the crossing and growing of steps (a) and (b) for an additional 3-10 generations to generate further ‘Spartan’-derived arugula plants.

In another embodiment, the present invention relates to a method of producing an arugula leaf from the plant of any of the above embodiments, said method comprising growing the plant of the above embodiments until it produces at least one leaf, and harvesting the leaf.

### BRIEF DESCRIPTION OF THE DRAWINGS

The patent or application file contains at least one drawing executed in color. Copies of this patent or patent application publication with color drawings will be provided by the office upon request and payment of the necessary fee.

FIGS. 1A-1B show arugula variety ‘Rock1’. FIG. 1A shows a whole plant of arugula variety ‘Rock1’. FIG. 1B shows the lower surface of a leaf of arugula variety ‘Rock1’.

FIGS. 2A-2B show arugula variety ‘Spartan’. FIG. 2A shows a whole plant of arugula variety ‘Spartan’. FIG. 2B shows the lower surface of a leaf of arugula variety ‘Spartan’.

### DETAILED DESCRIPTION

There are numerous steps in the development of novel, desirable arugula germplasm. Plant breeding begins with the analysis of problems and weaknesses of current arugula germplasms, the establishment of program goals, and the definition of specific breeding objectives. The next step is selection of germplasm that possess the traits to meet the program goals. The goal is to combine in a single variety or hybrid an improved combination of desirable traits from the parental germplasm. These important traits may include increased head size and weight, higher seed yield, improved color, resistance to diseases and insects, tolerance to drought and heat, and better agronomic quality.

Choice of breeding or selection methods can depend on the mode of plant reproduction, the heritability of the trait(s) being improved, and the type of variety used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

The complexity of inheritance influences choice of the breeding method. Backcross breeding is used to transfer one or a few favorable genes for a highly heritable trait into a desirable variety. This approach has been used extensively for breeding disease-resistant varieties. Various recurrent selection techniques are used to improve quantitatively inherited traits controlled by numerous genes. The use of recurrent selection in self-pollinating crops depends on the case of pollination, the frequency of successful hybrids from each pollination, and the number of hybrid offspring from each successful cross.

Each breeding program may include a periodic, objective evaluation of the efficiency of the breeding procedure. Evaluation criteria vary depending on the goal and objectives, and can include gain from selection per year based on comparisons to an appropriate standard, the overall value of the advanced breeding lines, and the number of successful varieties produced per unit of input (e.g., per year, per dollar expended, etc.).

Promising advanced breeding lines may be thoroughly tested and compared to appropriate standards in environments representative of the commercial target area(s) for at least three years. The best lines can then be candidates for new commercial varieties. Those still deficient in a few traits may be used as parents to produce new populations for further selection. These processes, which lead to the final step of marketing and distribution, may take from ten to twenty years from the time the first cross or selection is made.

One goal of arugula plant breeding is to develop new, unique, and genetically superior arugula varieties. A breeder can initially select and crosses two or more parental lines, followed by repeated selfing and selection, producing many new genetic combinations. Moreover, a breeder can generate multiple different genetic combinations by crossing, selfing, and mutations. A plant breeder can then select which germplasms to advance to the next generation. These germplasms may then be grown under different geographical, climatic, and soil conditions, and further selections can be made during, and at the end of, the growing season.

The development of commercial arugula varieties thus requires the development of parental arugula varieties, the crossing of these varieties, and the evaluation of the crosses. Pedigree breeding and recurrent selection breeding methods may be used to develop varieties from breeding populations. Breeding programs can be used to combine desirable traits from two or more varieties or various broad-based sources into breeding pools from which new varieties are developed by selfing and selection of desired phenotypes. The new varieties are crossed with other varieties and the hybrids from these crosses are evaluated to determine which have commercial potential.

Pedigree breeding is generally used for the improvement of self-pollinating crops or inbred lines of cross-pollinating crops. Two parents which possess favorable, complementary traits are crossed to produce an F₁. An F₂ population is produced by selfing one or several F₁'s or by intercrossing two F₁'s (sib mating). Selection of the best individuals is usually begun in the F₂ population. Then, beginning in the F₃, the best individuals in the best families are selected. Replicated testing of families, or hybrid combinations involving individuals of these families, often follows in the F₄ generation to improve the effectiveness of selection for traits with low heritability. At an advanced stage of inbreeding (i.e., F₆ and F₇), the best lines or mixtures of phenotypically similar lines are tested for potential release as new varieties.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Additional non-limiting examples of breeding methods that may be used include, without limitation, those found in Principles of Plant Breeding, John Wiley and Son, pp. 115-161 (1960); Allard (1960); Simmonds (1979); Sneep, et al. (1979); Fehr (1987); and “Carrots and Related Vegetable Umbelliferae,” Rubatzky, V. E., et al. (1999).

### Definitions

In order to more clearly understand the invention, the following definitions are provided:

Allele. The allele is any of one or more alternative forms of a gene, all of which relate to one trait or characteristic. In a diploid cell or organism, the two alleles of a given gene occupy corresponding loci on a pair of homologous chromosomes.

Backcrossing. Backcrossing is a process in which a breeder repeatedly crosses hybrid progeny back to one of the parents, for example, a first generation hybrid F₁ with one of the parental genotype of the F₁ hybrid.

Bolting. The premature development of a flowering stalk, and subsequent seed, before a plant produces a food crop. Bolting is typically caused by late planting when temperatures are low enough to cause vernalization of the plants.

Bremia lactucae. An oomycete that causes downy mildew in leafy vegetables, such as lettuce and arugula, in cooler growing regions.

Cotyledon. One of the first leaves of the embryo of a seed plant; typically one or more in monocotyledons, two in dicotyledons, and two or more in gymnosperms.

Essentially all the physiological and morphological characteristics. A plant having essentially all the physiological and morphological characteristics means a plant having the physiological and morphological characteristics of the recurrent parent, except for the characteristics derived from the converted gene.

First water date. The date the seed first receives adequate moisture to germinate. This can and often does equal the planting date.

Gene. As used herein, “gene” refers to a segment of nucleic acid. A gene can be introduced into a genome of a species, whether from a different species or from the same species, using transformation or various breeding methods.

Hyaloperonospora parasitica. An oomycete that causes downy mildew in leafy vegetables, such as arugula.

Maturity date. Maturity refers to the stage when the plants are of full size or optimum weight, in marketable form or shape to be of commercial or economic value.

Munsell: Munsell refers to the Munsell Color Chart, which uses the Munsell color system.

Quantitative Trait Loci. Quantitative Trait Loci (QTL) refers to genetic loci that control to some degree, numerically representable traits that are usually continuously distributed. Regeneration. Regeneration refers to the development of a plant from tissue culture.

Single gene converted. Single gene converted or conversion plant refers to plants which are developed by a plant breeding technique called backcrossing or via genetic engineering where essentially all of the desired morphological and physiological characteristics of a line are recovered in addition to the single gene transferred into the line via the backcrossing technique or via genetic engineering.

Taking into account these definitions, the present invention is directed to seeds of the arugula varieties ‘Rock 1’ and ‘Spartan’, plants produced by growing ‘Rock 1’ and/or ‘Spartan’ arugula seeds, heads isolated or harvested from the plants, one or more plants selected from a collection of ‘Rock 1’ and/or ‘Spartan’ plants and seeds derived or produced therefrom; plants produced by crossing an arugula plant with a ‘Rock 1’ and/or ‘Spartan’ arugula plant and seeds derived or produced therefrom.

### Overview of the Variety ‘Rock1’

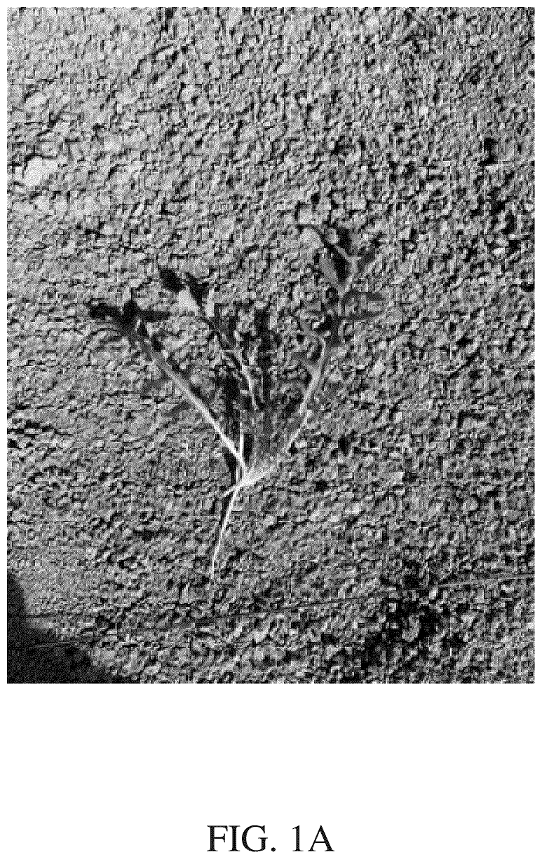

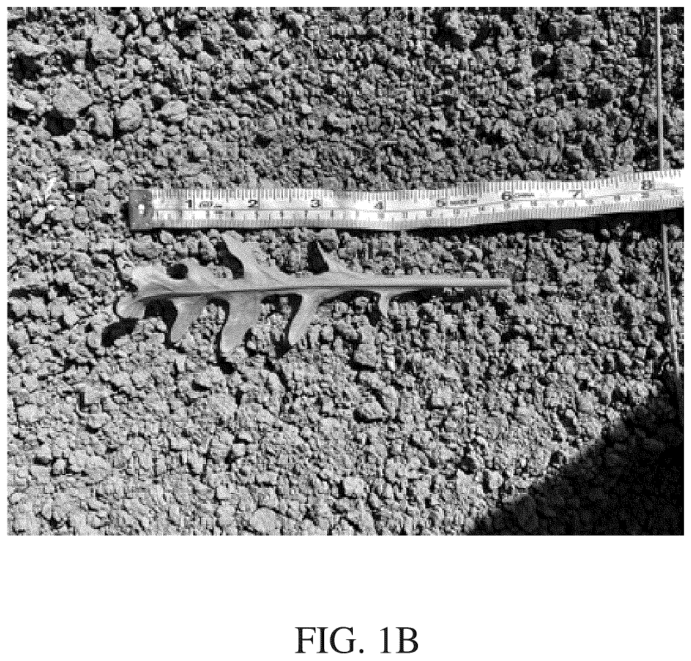

Arugula variety ‘Rock1’ is an Eruca sativa Mill. variety with dark green leaf blade color and very strong leaf division. FIGS. 1A-1B depict a whole plant and a leaf of arugula variety ‘Rock1’. Arugula variety ‘Rock1’ is the result of numerous generations of plant selections chosen for its very fast growth, slow bolting, dark green color, and distinctive lobing.

The variety has shown uniformity and stability for the traits, within the limits of environmental influence for the traits. It has been increased with continued observation for uniformity. No variant traits have been observed or are expected in variety ‘Rock1’.

### Objective Description of the Variety ‘Rock1’

The data which define these characteristics is based on observations taken in fields located in Yuma, Arizona, USA. Measurements of ‘Rock1’ were made 6 weeks after seeding. Arugula variety ‘Rock1’ has the following morphologic and other characteristics:

### Plant:

Plant height at flowering: 90 cm
Days from planting to prime market stage: 20-25

### Leaves:

Attitude: Erect
Color of blade: Munsell 5GY 4/4 (Dark green)
Intensity of color: Medium
Length, including petiole: 15 cm
Width: 4.5 cm
Division: Very strong
Secondary lobing: Strong
Undulation of margin: Weak
Hairiness: Weak

### Flower:

Time of flowering (50% of plants have at least one open flower): Late
Speed of bolting (elongation of flower stalk with or without flowers): Slow
Color of petals (at time of opening): Whitish
Anthocyanin coloration of veins: Absent or very weak

### Seed:

Seed color: Munsell 5YR 6/8 (Light-dark brown)

### Comparisons of ‘Rock1’ to Other Arugula Variety

Table 1 below compares characteristics of Eruca sativa variety ‘Rock1’ with the Eruca sativa varieties ‘Astro’ (unpatented) and ‘Slow Bolt’ (unpatented). Column 1 lists the characteristic, column 2 shows the characteristic for arugula variety ‘Rock1’, column 3 shows the characteristic for arugula variety ‘Astro’, and column 4 shows the characteristic for arugula variety ‘Slow Bolt’.

TABLE 1

| Characteristic | ‘Rock1’ | ‘Astro’ | ‘Slow Bolt’ |
|---|---|---|---|
| Lobing | Strong | Weak | Weak |
| Bolting | Slow | Fast | Slow |
| Leaf color | Dark Green | Pale Green | Pale Green |

### Overview of the Variety ‘Spartan’

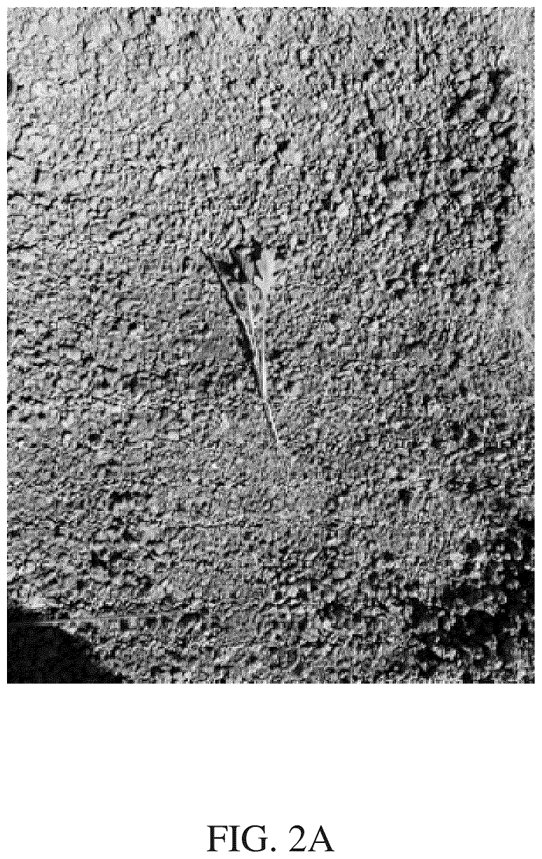

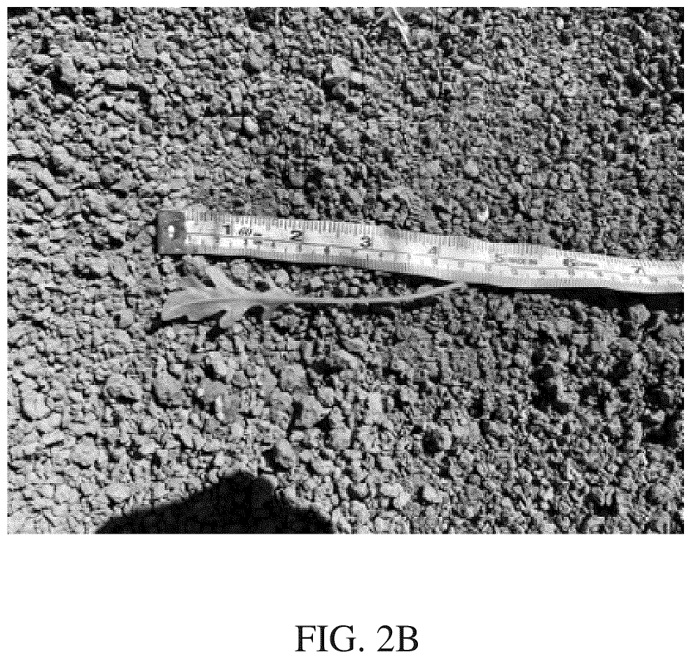

Arugula variety ‘Spartan’ is a Diplotaxis tenuifolia Mill. variety with medium green leaf blade color and very strong leaf division. FIGS. 2A-2B depict a whole plant and a leaf of arugula variety ‘Spartan’. Arugula variety ‘Spartan’ is the result of numerous generations of plant selections chosen for its medium fast growth, very slow bolting, dark green color, and distinctive lobing.

The variety has shown uniformity and stability for the traits, within the limits of environmental influence for the traits. It has been increased with continued observation for uniformity. No variant traits have been observed or are expected in variety ‘Spartan’.

### Objective Description of the Variety ‘Spartan’

The data which define these characteristics is based on observations taken in fields located in Yuma, Arizona, USA. Measurements of ‘Spartan’ were made 6 weeks after seeding. Arugula variety ‘Spartan’ has the following morphologic and other characteristics:

### Plant:

Plant height at flowering: 90 cm
Days from planting to prime market stage: 40-45

### Leaves:

Attitude: Semi-erect
Color of blade: 5GY 5/4 (Medium green)
Length, including petiole: 10.5 cm
Width: 2.3 cm
Division: Very strong
Width of primary lobes: 1.5 cm
Secondary lobing: Strong
Undulation of margin: Medium
Hairiness: Weak

### Flower:

Time of flowering (50% of plants have at least one open flower): Very late
Speed of bolting (elongation of flower stalk with or without flowers): Very slow
Color of petals (at time of opening): Light yellow
Anthocyanin coloration of veins: Weak

### Seed:

Seed color: 5YR 6/10 (Light brown)

### Comparisons of ‘Spartan’ to Other Arugula Varieties

Table 2 below compares characteristics of Diplotaxis tenuifolia variety ‘Spartan’ with the Eruca sativa variety ‘Apollo’ (unpatented) and Diplotaxis tenuifolia variety ‘Extrema’ (unpatented). Column 1 lists the characteristic, column 2 shows the characteristic for arugula variety ‘Spartan’, column 3 shows the characteristic for arugula variety ‘Apollo’, and column 4 shows the characteristic for arugula variety ‘Extrema’.

TABLE 2

| Characteristic | ‘Spartan’ | ‘Apollo’ | ‘Extrema’ |
|---|---|---|---|
| Uniformity | High | Average | High |
| Flowering | Very slow | Medium | Medium |

### Further Embodiments

### Genotyping

In addition to phenotypic observations, the genotype of a plant can also be examined. There are many laboratory-based techniques known in the art that are available for the analysis, comparison and characterization of plant genotype. Such techniques include, without limitation, DNA- or RNA-sequencing, CAPS Markers, ELISA, Western blot, microarrays, Single Nucleotide Polymorphisms (SNPs), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Differential Display Polymerase Chain Reaction (DD-PCR), Quantitative Real-Time Polymerase Chain Reaction (qRT-PCR), DNA Amplification Fingerprinting (DAF), Sequence Characterized Amplified Regions (SCARs), Amplified Fragment Length Polymorphisms (AFLPs), and Simple Sequence Repeats (SSRs, which are also referred to as Microsatellites).

### Deposit Information

### Arugula Variety ‘Spartan’

A deposit of at least 625 seeds of the Diplotaxis tenuifolia Mill. variety ‘Spartan’ was made with the American Type Culture Collection (ATCC), ATCC Patent Depository, 10801 University Boulevard, Manassas, Virginia, 20110, USA, and assigned ATCC number X2. The seeds deposited with the ATCC on DATE were obtained from the seed of the variety maintained by Pinnacle Seed, Inc., P.O. Box 222672, Carmel, California 93923, United States of America since prior to the filing date of the application. Access to this deposit will be available during the pendency of this application to persons determined by the Commissioner of Patents and Trademarks to be entitled thereto under 37 C.F.R. § 1.14 and 35 U.S.C. § 122. Upon issuance, the Applicant will make the deposit available to the public consistent with all of the requirements of 37 C.F.R. § 1.801-1.809. This deposit of the arugula variety ‘Spartan’ will be maintained in the ATCC, which is a public depository, for a period of 30 years, or at least 5 years after the most recent request for a sample of the deposit, or for the effective life of the patent, whichever is longer, and will be replaced if it becomes nonviable during that period. Applicant has no authority to waive any restrictions imposed by law on the transfer of biological material or its transportation in commerce. Applicant does not waive any infringement of rights granted under this patent or under the Plant Variety Protection Act (7 USC 2321 et seq.).

What is claimed:

1. A Diplotaxis tenuifolia Mill. arugula seed designated as ‘Spartan’, representative sample of seed having been deposited under ATCC Accession Number X2.

2. An arugula plant produced by growing the seed of claim 1.

3. A plant part from the plant of claim 2, wherein said part is a leaf, an ovule, a pollen grain, a seed, an embryo, an embryo sac, a fruit, a cell, a callus, a protoplast, a root, a rootstock, a stem, a meristem, a cotyledon, a hypocotyl, a flower, an ovary, a pericarp, an anther, a microspore, or a portion thereof.

4. The plant part of claim 3, wherein said part is a stem, a leaf, or a portion thereof.

5. An arugula plant having all the physiological and morphological characteristics of the arugula plant of claim 2.

6. A plant part from the plant of claim 5, wherein said part is a leaf, an ovule, a pollen grain, a seed, an embryo, an embryo sac, a fruit, a cell, a callus, a protoplast, a root, a rootstock, a stem, a meristem, a cotyledon, a hypocotyl, a flower, an ovary, a pericarp, an anther, a microspore, or a portion thereof.

7. The plant part of claim 6, wherein said part is a stem, a leaf, or a portion thereof.

8. An F₁ hybrid arugula plant having ‘Spartan’ as a parent, where ‘Spartan’ is grown from the seed of claim 1.

9. A pollen grain or an ovule of the plant of claim 2.

10. A tissue culture of the plant of claim 2.

11. A tissue or cell culture produced from protoplasts or cells from the plant of claim 2, wherein said cells or protoplasts are produced from a plant part selected from the group consisting of root, root tip, meristematic cell, stem, hypocotyl, petiole, cotyledon, leaf, flower, anther, pollen, pistil, embryo, seed, and fruit.

12. An arugula plant regenerated from the tissue culture of claim 11, wherein the plant has all of the morphological and physiological characteristics of an arugula plant produced by growing seed designated as ‘Spartan’, representative sample of seed having been deposited under ATCC Accession Number X2.

13. A method of making arugula seeds, said method comprising crossing the plant of claim 2 with another arugula plant and harvesting seed therefrom.

14. The method of claim 13, wherein the method further comprises:
(a) selecting one or more of the F₁ progeny for plants that have a desired trait to produce selected progeny plants;
(b) crossing the selected progeny plants with a variety of arugula plant designated ‘Spartan’, representative sample of seed of ‘Spartan’ having been deposited under ATCC Accession Number X2, to produce backcross progeny plants;
(c) selecting for backcross progeny plants that have the desired trait and all of the physiological and morphological characteristics ‘Spartan’ arugula when grown in the same environmental conditions to produce selected backcross progeny plants; and
(d) repeating steps (b) and (c) three or more times in succession to produce selected fourth or higher backcross progeny plants that comprise the desired trait and all of the physiological and morphological characteristics of ‘Spartan’ arugula when grown in the same environmental conditions.

15. A method of producing a seed of a ‘Spartan’-derived arugula plant, comprising crossing a Diplotaxis tenuifolia Mill. plant designated as ‘Spartan’, representative sample of seed having been deposited under ATCC Accession Number X2, with a second Diplotaxis tenuifolia Mill. plant, whereby seed of a ‘Spartan’-derived arugula plant forms.

16. The method of claim 15 further comprising:
(a) crossing a plant grown from Diplotaxis tenuifolia Mill. seed derived from ‘Spartan’ with itself or with a second Diplotaxis tenuifolia Mill. plant to yield additional seed of a ‘Spartan’-derived arugula plant;
(b) growing the additional seed of a ‘Spartan’-derived arugula plant of step (a) to yield additional ‘Spartan’-derived arugula plants; and
(c) repeating the crossing and growing of steps (a) and (b) for an additional 3-10 generations to generate further ‘Spartan’-derived arugula plants.

17. A method of producing an arugula leaf, said method comprising growing the plant of claim 2 until it produces at least one leaf, and harvesting the leaf.
